(19) United States
(12) Patent Application Publication
(10) Pub. No.: US 2006/0205093 A1
(43) Pub. Date: Sep. 14, 2006

(54) Use of magnetic particles for determining binding between bioactive molecules

(75) Inventor: Menno Willem Jose Prins, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: Koninklijke Philips Electronics N.V., Eindhoven (NL)

(21) Appl. No.: 10/566,555

(22) PCT Filed: Jul. 14, 2004
(86) PCT No.: PCT/IB04/51213

§ 371 Date: Jan. 27, 2006

(30) Foreign Application Priority Data
Jul. 30, 2003 (EP) 03102352.6

(51) Int. Cl. G01N 33/553

(52) U.S. Cl. 436/526

(57) ABSTRACT

An assay as well as tools and apparatus therefore are disclosed for determining interaction between microbiological entities such as bioactive molecules using at least a first particle or microcarrier e.g. a bead, and a second particle which may also be a microcarrier, e.g. a second bead. At least the first microcarrier is magnetic. When two beads are used and both beads are magnetic, the beads preferably differ in the size of their magnetic moment. A means is provided for placing a binding between bioactive molecules under a mechanical stress to thereby distinguish between bindings of different strengths. In one aspect, the second bead, (with a larger magnetic moment) is used to magnetically remove target molecules linked to beads with smaller magnetic moment which are weakly bound to a capture molecule (itself generally coupled to a mobile or immobile surface). Alternatively, fluid frictional forces can be applied to one of the particles to disrupt weak bindings. Depending upon the embodiment, the first bead and/or second particle can be used for detection purposes.

The present invention describes methods, apparatus and tools for determining interactions between microbiological entities such as viruses, protozoa, bacteria, organelles thereof, liposomes and bioactive molecules such as proteins or DNA. The present invention in particular presents solutions for discriminating between a-specific and specific binding between a microbiological entity such as a bioactive molecule and another entity. The invention has applications in the design and use of biochemical and medicinal diagnostic assays, such as protein microarrays.

The challenge of biosensing is to detect small concentrations of specific target molecules (e.g. tumor markers in the pmol/l range and lower) in a complex mixture (e.g. blood) with high concentrations of background material (e.g. mmol/l of albumin). The most common biosensing method is to coat a surface with capture molecules (e.g. antibodies, nucleic acids, etc.). These molecules capture the targets which are subsequently detected. The detection of target molecules can be performed with or without a label. The labeling step can occur before or after the capture on the surface. The label can be directly coupled to the target, or indirectly, e.g. via another bio-active molecule. Most frequently optical labels are used for detection, e.g. fluorescent molecules.

An important problem in biosensing is non-specific binding of a detection label or a target molecule, to the surface or to the capture molecules of the biosensor. This generates a background signal, which reduces the analytical sensitivity and specificity of the biosensor. In diagnostic assays, non-specific binding can be reduced by using stringency procedures. Stringency procedures aim to release unwanted a-specific adsorption binding, and only maintain the specific interactions between capture molecules and (labeled) target molecules. The most common stringency method is a washing step. The composition and temperature of the washing solution is carefully tuned to reduce the background for a given assay.

There is an increasing focus on multi-analyte detection, where many different molecules are simultaneously measured on a single surface, called a biochip or (micro)array. The biochip surface is composed of many spots (“capture spots”), every spot comprising a different capture molecule or molecules. It is difficult to develop stringent whole-chip washing steps for multi-analyte sensors, because a multitude of possible background signals need to be suppressed, while a range of different specific interactions has to remain undisturbed, and the native state of different target and capture molecules has to be preserved. The result is a lower analytical sensitivity and a lower analytical specificity. The multi-analyte stringency problem is very serious for protein detection, because proteins and protein-protein interactions are very heterogeneous, and the effect of a certain washing step on the denaturation of proteins and on protein-protein interaction can be very significant.

This major drawback of protein arrays or microarrays has been recognized in the article of Macbeatch and Schreiber (2000) in Science 289, 1760-1763, describe the application of native proteins on microscopic glass slides. A-specific protein-protein binding is reduced by adding BSA on the capture molecules, prior to the binding with target molecules. A review article of December 2002 (Macbeath (2002) Nature Genetics 32, S526-532) on protein microarray technology mentions the problem of assessing specificity as a key feature of protein array technology without suggesting potential solutions. The most successful applications of protein array technology up to now are achieved in the field of immunology. The high binding strength between antigen and antibody allows stringent washing conditions to overcome a-specific interactions of antigen and antibody.

A chemical way to address the stringency problem is by designing capture molecules that can interact more strongly and more specifically with the targets. An example is photo-aptamers, synthetic capture molecules that carry photo-reactive groups which can cross-link at specific sites of the target molecule (reviewed in Brody & Gold (2000) J. Biotechnol. 7, 5-13). When a capture surface comprising photo-aptamers is exposed to the sample and a photo-excitation step is applied, molecules that fit the aptamer binding site become covalently linked thereto. Subsequently a severe washing step can be applied to remove molecules that have not photo-reacted. Disadvantages of the photo-aptamer approach are that it requires the design of a new photoaptamer for every target, that a photo-excitation step is required, that the photo-crosslinking step itself does not distinguish between specifically and a-specifically bound molecules, and that it is an endpoint detection method.

US application No. 2002/0001855 (Prentiss application) describes a method for detecting the binding strength between two molecules wherein one of the molecules is coupled to a first surface and brought in contact with the other molecule, which is magnetically labeled. The first surface then is brought in proximity to a second surface having a magnetic field, which will exert a pulling force on the magnetic labels and, depending on the strength of the binding between the two molecules, result in a separation thereof. The main disadvantage of this system is the physical distance between the second surface and the magnetic label (present on the first surface) which limits the sensitivity of the assay. The second surface is a device at which magnetic field gradients are generated. The device is made as a grid of small magnets. As a result, the device has regions of high magnetic field gradient near the grid surface. The magnitude of the local gradients and the distance to which the high-gradient region extends outside the device are determined by the magnetic field generated by the elements and the size of the magnetic elements of which the grid is composed. A disadvantage of the device is that field gradients rapidly drop as a function of distance. Thus, high gradients are only present very close to the grid surface. Therefore, the device has to be brought into very close proximity to the biological binding surface, which is generally not possible due to the presence of sample fluid and the presence of a fluid chamber with a cover plate above the biological binding surface.

There is a need for the design of improved methods, apparatus and tools for determining the interaction or binding between microbiological entities such as bioactive molecules, especially for methods wherein a discrimination can be made between bindings of different strengths such as between specific and a-specific binding. The discrimination between specific and a-specific binding is especially needed with multi-analyte sensors where it should simultaneously be applicable to a wide range of capture and target molecules. There is a need for protein multi-analyte sensors since modifying buffer conditions to improve stringency on the multi analyte sensor can only be done within very narrow limits. Further, in order for the method to be of practical use in micro-array set-ups, there is a need for downscaling without loss of sensitivity.

An object of the present invention is to provide alternative methods, apparatus and tools for determining interactions between microbiological entities such as bioactive molecules, for instance proteins or DNA or between bioactive molecules and microbiological entities such as viruses, bacteria, protozoa, liposomes, or fragments thereof, especially those which meet at least one need as mentioned above.

The present invention discloses an assay as well as tools and apparatus therefore, for determining interaction between bioactive molecules or other microbiological entities using at least one particle or microcarrier e.g. a bead or a microbiological entity, which is magnetic and a second particle which may also be a microcarrier, e.g. a second bead or microbiological entity. At least the first particle or microcarrier is magnetic. When two beads are used and both beads are magnetic, the beads preferably differ in the size of their magnetic moment. Means are provided for allowing conditions in a liquid to promote bindings between bioactive molecules or between bioactive molecules and other microbiological entities or between microbiological entities and to place the bindings under a mechanical stress to thereby distinguish between bindings of different strengths. In one aspect, for example, the second bead, (with a larger magnetic moment) is used to magnetically remove target molecules linked to beads with a smaller magnetic moment which are weakly bound to a capture molecule (itself generally coupled to a mobile or immobile surface). In accordance with other embodiments a mechanical frictional force, e.g. as caused by fluid flow, is imposed on at least one of the particles to thereby induce a mechanical stress in the binding. Depending upon the embodiment, the first bead and/or second particles can be used for detection purposes.

Obviously, the first and/or second particle can be an individual particle, but it can also be part of a collection of particles, e.g. a cluster of particles or a chain of particles.

Chains of particles are easily formed in the presence of a magnetic field. These chains of particles are easily transferred in a liquid. Moreover, said chains of particles have a strong field gradient at their ends, which is favorable for stringency properties.

In one aspect the present invention provides a method for distinguishing between different strengths of bindings between microbiological entities in a liquid, using a first and a second particle, at least one of which is magnetic, in a magnetic field, the use comprising: providing a complex between a first particle mobile in the liquid and a first microbiological entity, providing conditions within the liquid for a binding between the first microbiological entity and a second microbiological entity; bringing a second particle mobile in the liquid into proximity with the complex; and acting on the first and/or second particle to apply a mechanical stress to the binding between the first and second microbiological entity while applying the magnetic field to thereby disrupt a binding of a first strength and not to disrupt a binding of a second greater strength. In embodiments of the present invention forces are applied directly to the first and second particle to generate a relative force between the particles. This relative force then generates or induces a mechanical stress in the binding. The first and second particles should have a mechanical rigidity such that the generated relative force between the particles does not disrupt the particles but rather the binding. Hence, the particles may comprise a rigid core, scaffold or matrix. The distinguishing of the strength of a binding may be used for the discrimination between a specific and a-specific binding. The first microbiological entity may be a target molecule and the second microbiological entity be a capture molecule, e.g. a capture molecule able to capture the target when both are in a liquid. Both first and second particles can be magnetic particles or only one needs to be magnetic. The first magnetic particle can be coupled to a microbiological entity and the second magnetic particle need not coupled to a microbiological entity. The first magnetic particle can be coupled to a target microbiological entity. The magnetic property of the first magnetic particle may be used to concentrate the target, e.g. by placing a field gradient on the liquid and attracting the magnetic particles bound to the target to a specific location. The first and/or second magnetic particles can be paramagnetic or any other magnetic form.

The first magnetic particle may have a magnetic moment which is smaller, e.g. 10 times smaller than the magnetic moment of the second magnetic particle. Alternatively, the first and the second particles are both magnetic and have the same magnetic moment. The size of the first magnetic particle can be smaller than the size of the second magnetic particle. The first magnetic particle preferably has a diameter in the range 1 nm to 1 μm, e.g. between 10 nm and 200 nm. The second magnetic particle can have a diameter of at least 100 nm.

The first or second microbiological entity can be any suitable entity but a protein or a peptide is preferred in some embodiments. Typically, more than one first microbiological entity and/or more than one second microbiological entity occur in the sample. The first or second microbiological entity can be arranged on capture spots on an array. This is convenient for detection purposes. For example, the array can comprise at least 10 different capture microbiological entities on different capture spots. A capture spot can occupy a space between 0,1 and 10⁴ μm².

In some embodiments of the present invention both the first and the second particles are coupled to a microbiological entity. For example, the first particle can be coupled to a target microbiological entity and the second particle is then coupled to a capture microbiological entity. Alternatively, the first particle can be coupled to a first target microbiological entity and the second particle can be coupled to a second target microbiological entity. The first or second microbiological entity can be an antibody. Monoclonal antibodies can provide a high level of specificity to certain proteins.

In another aspect only one of the first and second particles is magnetic and the other particle is non-magnetic. The non-magnetic particle can be larger than the magnetic particle. The present invention includes applying a fluid frictional force to the first or second microbiological entity. In such a case, the fluid friction force applied to the second particle is much larger than to the first particle due to the size difference. Different sizes of non magnetic particles can be used in order to distinguish between different strengths of bindings.

The magnetic field which is applied can be between 1.10⁻⁴ and 10 Tesla, e.g. between 0,01 and 0,1 Tesla Where the first and second particles are magnetic, the magnetic field can create an attractive or a repulsive force between the first and the second particle or a varying, oscillating or alternating force.

The present invention also includes contacting the bound first and second microbiological entity with a third magnetic particle, wherein the third magnetic particle has a magnetic moment which is larger than the magnetic moment of the first or second magnetic particle.

The present invention also provides a method of using a first and a second particle for distinguishing between a first and a second binding between capture and target microbiological entities such as capture and target bioactive molecules, the first binding having a different strength than the second binding, wherein at least one of the particles is magnetic, the method comprising: contacting a sample with at least one capture microbiological entity with at least one target microbiological entity coupled to a first particle, providing conditions for allowing a binding between a capture and target microbiological entities, in the case of an uncoupled capture microbiological entity, contacting the sample with an uncoupled second particle or contacting the sample with a second particle coupled to a microbiological entity, applying a magnetic field, distinguishing between whether the binding between the target and the capture microbiological entity is a first or second binding.

The present invention also provides an apparatus or a tool for distinguishing between bindings of different strengths between microbiological entities in a liquid, the tool comprising: first particles and second particles, at least one of which is magnetic, both first and second particles being mobile in a liquid; means acting on the first and second particles to thereby exert a mechanical stress on bindings between the first and second microbiological entities and to distinguish between the bindings of different strengths, the means for exerting a mechanical stress comprising at least a magnetic field generator. The means for exerting a mechanical stress may include means for exerting a fluid frictional force on the first or second particles. The tool may further comprise an array of microbiological entities arranged on capture spots on a substrate. The tool may also comprise means for generating an excitation that forces a lateral movement of the particles with respect to the array.

The tool may be used for the identification, isolation, purification of a specific bound bioactive molecule. The tool is preferably fabricated as a microfluidic device.

The dependent claims define individual embodiments of the present invention.

The present invention will now be described with reference to the following drawings.

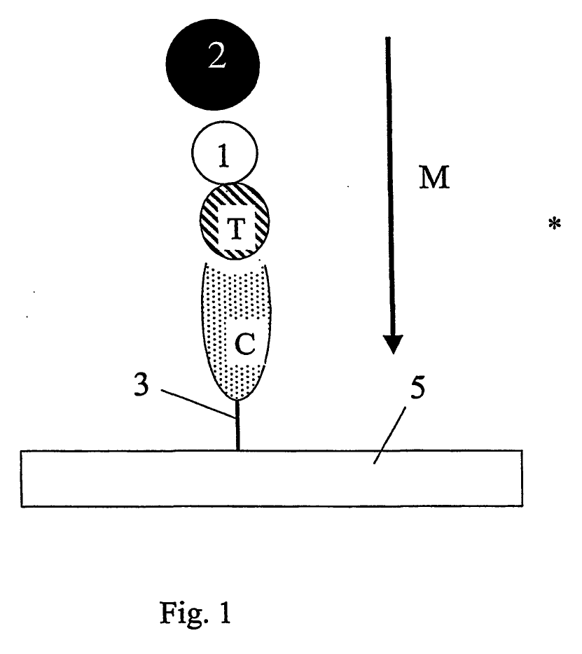

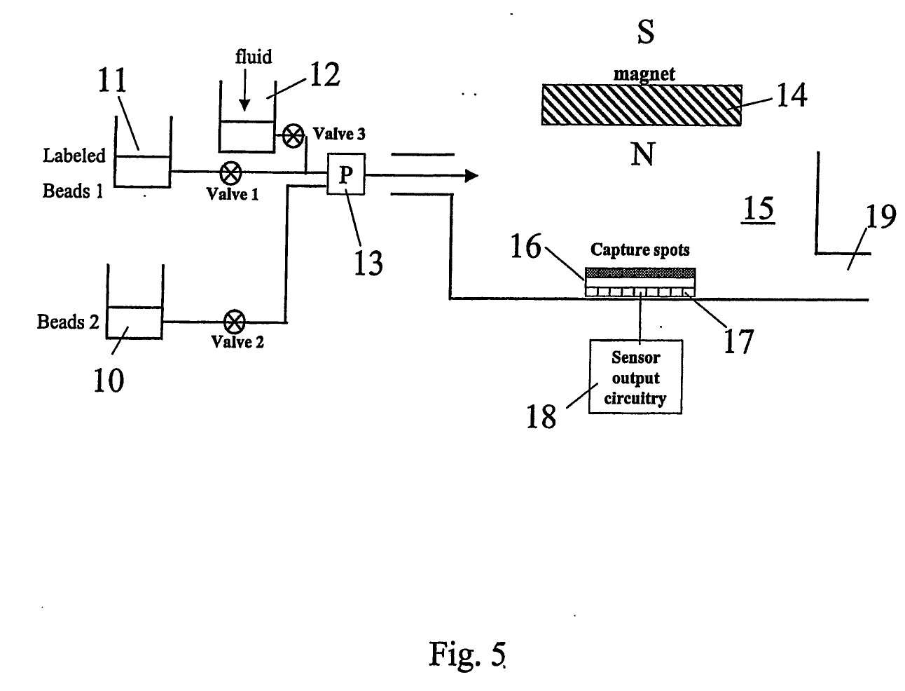

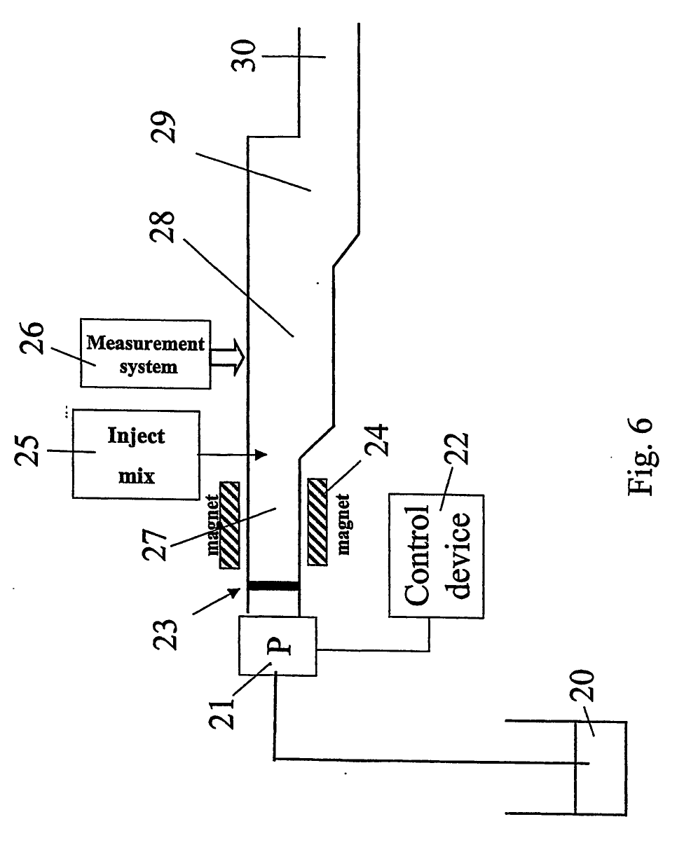

FIGS. 1-4 show different embodiments of the present invention while FIGS. 5 and 6 refer to prior art. In all Figs. beads are represented by open circles (1) and closed filled circles (2). Capture molecules (C) are dotted, target molecules (T) are lined. The configuration shown refers to the situation where capture and target molecules are bound to each other and beads have migrated close enough to perform a disruptive force on the target capture complex.

FIG. 1 represents, according to an embodiment of the invention, a detail of a micro array wherein a second magnetic bead (2) performs an attractive force on the magnetic bead (1) coupled to a target molecule.

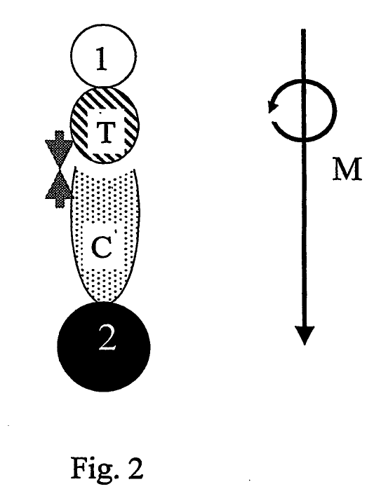

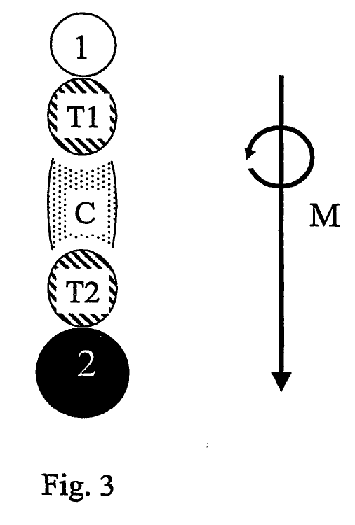

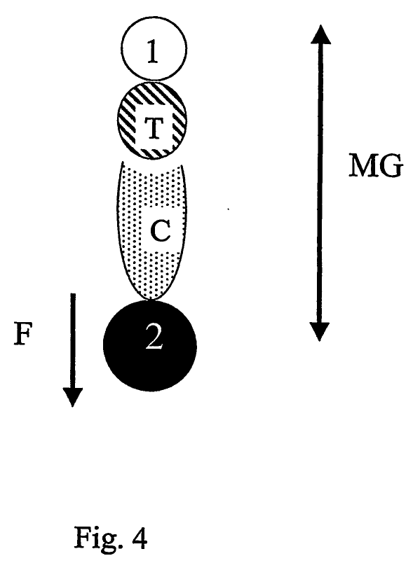

FIGS. 2-4 represent, according to embodiments of the invention, target molecules bound to capture molecules, wherein a second bead (2) performs a repulsive force on a magnetic bead (1). In FIG. 2 and FIG. 3, respectively one and 2 target molecules are bound to a capture molecule. In FIG. 4 the second bead (2*) is a non magnetic bead and the repulsive forces are a magnetic force and a fluid friction force (indicated with *).

FIG. 5 is a schematic drawing of an apparatus in accordance with an embodiment of the present invention able to perform the method of FIG. 1 or 2.

FIG. 6 is a schematic drawing of an apparatus in accordance with another embodiment of the present invention able to perform the method of FIG. 4.

The present invention will be described with respect to particular embodiments and with reference to certain drawings but the invention is not limited thereto but only by the claims. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

Moreover, the terms top, bottom, over, under and the like in the description and the claims are used for descriptive purposes and not necessarily for describing relative positions. It is to be understood that the terms so used are interchangeable under appropriate circumstances and that the embodiments of the invention described herein are capable of operation in other orientations than described or illustrated herein.

The present invention is based on the observation that for the detection of interactions between microbiological entities (e.g. between viruses, bacteria, protozoa and bioactive molecules such as antibodies. The interactions will generally be protein-protein or peptide-peptide interactions) or between bioactive molecules (e.g. peptide-peptide or protein-protein interactions) in general and for multi-analyte sensors in particular, it would be advantageous to remove weakly or non-specifically bound molecules by a physical (stringency) force that is independent of the precise physicochemical nature of the capture molecule-target interaction. More generally, it is the aim of the present invention to discriminate between weak and strong interactions between bioactive molecules or microbiological entities or to distinguish between a first interaction and a second weaker interaction.

The minimum force that the method should be able to apply can be estimated from F_min=E_min/w_max, with E_min the lowest energy of interactions and w_max the largest length scale of interactions. E_min is given by the thermal energy at which the interactions between the bioactive molecules or other microbiological entities occur. The value of w_max is determined by the maximum interaction range of biological interactions (e.g. ionic, van der Waals, hydrogen bonds, hydrophobic/philic, steric). With E_min=k_{b}T, T=300 K and w_max=40 nm, a minimum force of 0.1 pN is the result which is achieved by the embodiments of the present invention.

The present invention provides a physical method to suppress background signals in multi-analyte biochips. Two types of particles are involved, for example two types of magnetic particles differing in their magnetic moment by a significant amount, e.g. one or several orders of magnitude: (i) first beads or microbiological entities such as ferritin-like magnetic proteins or magnetosomes which may be smaller in size as part of the detection label and (ii) second beads or microbiological entities such as ferritin-like magnetic proteins or magnetosomes or magnetic cells such as magnetospirrillum magnetotactum. For example when beads are used the second beads may be larger in size to attract and transport small beads. Forces between the large and small particles are in the Pico Newton range, sufficient to make a distinction between strong and weak bio-molecular interactions. The method is applicable to a vast range of detection principles and device designs. Chemical and physical stringency methods can be combined with the present invention.

According to the present invention, a practical way to apply a physical force relies on at least one magnetic or paramagnetic particle or microcarrier, e.g. a bead or a microbiological entity. In accordance with the present invention a particle or microcarrier may be of any shape, e.g. in the form of spheres, cylinders or rods, cubes, ovals etc. or may have a variable shape such as a bacteria, magnetosome, liposome or protozoa. When magnetic microcarriers or particles are coupled to bioactive molecules, a force can be applied via a magnetic field gradient. The force F on a magnetic microcarrier or particle equals:
F=∇(m.B)≅m∇B Eq. (1)
with m the magnetic moment of the microcarrier or particle and B the magnetic field. The right-hand approximation applies for a constant particle moment, caused for example by magnetic saturation.

According to one aspect of the present invention, the magnetic force is generated using a combination of a first microcarrier or particle, e.g. a label such as a bead, and a second microcarrier or particle such as a second bead, for example a second magnetic or non-magnetic bead, which can be brought into close contact with the first magnetic bead.

In the following the present invention will mainly be described with reference to the use of beads. However, the present invention is not limited to the use of beads. In the following reference will be made to a ‘first bead’ and a ‘second bead’ however no limitation should be interpreted into the use of “fisrt” or “second” nor to the term “bead”. “Bead” as used in this invention does not mean that the bead is spherical in shape but may be of any suitable shape, e.g. in the form of spheres, cylinders or rods, cubes, ovals etc. or may have no defined or constant shape. Also, although the term “bead” is used in the following, the present invention includes the use of particles such as microbiological entities, e.g. magnetosomes, bacteria, viruses, protozoa, liposomes, protein complexes as equivalents of the mentioned beads, either magnetic or non-magnetic. For example, a magnetosome is a magnetic particle naturally occurring in magnetic bacteria and isolatable therefrom and may serve as a magnetic bead whereas a liposome is typically a synthetic particle which may be used as a non-magnetic bead.

Similar considerations apply to the term “bioactive molecule”. In the following reference will mainly be made to bioactive molecule, however the present invention includes the use of microbiological entities such as magnetosomes, bacteria, viruses, protozoa, liposomes, or fragments of any of these as well as protein complexes as equivalents of the mentioned molecules. The term “microbiological entities” should be interpreted broadly. It includes bioactive molecules such as proteins, peptides, RNA, DNA, lipids, phospholipids and carbohydrates or similar. The term bioactive molecules also includes cell fragments such as portions of cell membranes, particularly portions of cell membranes which may contain a receptor. The term bioactive molecules also relates to small compounds which potentially can bind a bioactive molecule. Examples herefore are ligands, agonists, antagonists, inhibitors, or modulators. The bioactive molecules can be isolated or synthesized molecules. Synthesized molecules can include non-naturally occurring compounds such as modified aminoacids or nucleotides. Alternatively, the bioactive molecules can occur within the context of lysate, a cell fraction, an organelle, an intact cell or an organism (e.g. virus or bacterium). The bioactive molecules can also occur in a medium such as blood or serum or other body fluids or secretions, or any other sample comprising bioactive molecules such as food, water samples and others. The term “microbiological entity” may also include viruses, bacteria, protozoa and other cellular organisms, or fragments thereof, or parts or organelles of cellular organisms such as magnetosomes, or synthetic microbiological bodies such as liposomes.

Interaction between molecules refers to binding and includes weak binding (generally a-specific or non-specific) and strong binding (generally specific binding). The terms “weak”, “a-specific” and “strong”, “specific” binding do not necessarily relate to a certain degree or absolute strength of binding but rather define a relative relationship, that is that the “strong” or “specific” has a stronger binding than the “weak” or “non-specific” or a-specific. Weak binding between bioactive molecules is understood as a binding with forces below 0.1 pN.

The interactions being determined in the present invention between the bioactive molecules referred to as a ‘target’ and a ‘capture molecule’ can be between any of the listed bioactive molecules mentioned and include but are not limited to receptor/ligand, receptor/inhibitor, enzyme/substrate, antibody/antigen, DNA/RNA, RNA/RNA, virus/molecule, bacteria/moleculee, liposome/molecule etc. combinations. Preferred types of interaction being determined are those wherein at least one of the target or the capture molecules is a protein or a peptide. More preferred types of interactions are those wherein both target and capture molecules are proteins or peptides.

Interactions between molecules and/or biological entities involving at least one binding should be given a broad interpretation in accordance with the present invention. The following complexes are given only as examples:

target molecules bound to capture molecules,

first target molecule bound to capture molecule bound to second target molecule,

capture molecules bound to a virus,

first capture molecule bound to virus bound to second capture molecule,

virus/cell molecule complexes.

By selecting the correct environment for the two particle system of which one particle is magnetic an increase in sensitivity can be obtained. The use of a magnetic bead to isolate and concentrate bioactive molecules is well known (e.g. B. Sinclair, “To bead or not to bead: Applications of magnetic bead technology”, The Scientist 12(13), p. 17, 22 June 1998; or “The Immunoassay Handbook” by David Wild (Nature Publishing Group, London, 2001, ISBN 1-56159-270-6); or Urs Häfeli et al, “Scientific and Clinical Applications of Magnetic Carriers” (Plenum Press, New York, 1997), ISBN: 0-306-45687-7) In these applications a capture molecule is coupled to a magnetic bead whereafter a magnetic force is applied to remove or concentrate the bound target. Usually these experiments are performed with beads having a size of 1 μm or larger, which is considerably larger than the average size of 10 nm of a protein. Though these beads can be used in the context of the present invention, their size may provide a significant drawback in a micro-array set-up due to steric hindrance. Steric hindrance limits the dynamic range of the sensing as well as the downscaling of the capture-spot. Downscaling is desired for capture spots, because a smaller capture spot with a higher density of capture molecules will result in a higher target concentration per unit area, and provide a greater signal-to-noise ratio of the sensing. Thus, this will allow obtaining the required sensitivity in a micro-array.

In one aspect, at least the first bead is attached to a bioactive molecule and the second bead is not attached to a bioactive molecule. However, according to a particular embodiment of the invention, a bioactive molecule can be attached to both the first and the second bead. Most preferably, in this case, the bioactive molecules will not be the same.

The beads, at least one of which is magnetic or paramagnetic, are used in such a way that application of a magnetic field or of other types of force field will result in a removal of non-specifically bound targets in a target-capture molecule interaction. The term “magnetic” includes any suitable form of magnetic particles, e.g. magnetic, paramagnetic, superparamagnetic, ferromagnetic, that is any form of magnetism which generates a magnetic dipole in a magnetic field, either permanently or temporarily. For performing the present invention there is no limitation on the shape of the beads, but spherical particles are the presently easiest and cheapest to manufacture in a reliable way. To improve the magnetic attraction, the beads may be provided with magnetic irregularities, e.g. high-curvature particles added on the surface, protrusions, needles, etc. Beads can also have a permanent magnetic moment e.g. due to a non-spherical shape. Preferred magnetic beads of the present invention are superparamagnetic particles. These generally consist of one or many small spherical cores (3-30 nm) of ferromagnetic material. Due to their small size, the cores consist of a single magnetic domain. In the case of superparamagnetic beads, a magnetic moment is generated by an external field. Due to the spherical shape, the magnetic moment can easily rotate inside the particle. Superparamagnetic particles have the advantage that their magnetic moment disappears in absence of an applied field, which then minimizes magnetic aggregation. An advantage of particles with a non-spherical shape and/or larger cores, is that the magnetic moment is generally large, which facilitates magnetic manipulation. Aggregation of particles can be removed by applying excitation methods such as alternating magnetic fields.

According to an embodiment of the invention, both of the beads are magnetic beads, most preferably beads with different magnetic moments. A set of beads wherein one bead is not magnetic (magnetic moment=0) is also included within this aspect of the invention. According to another embodiment of the invention (as described in the examples) the two beads can have the same magnetic moment.

According to one aspect of the present invention, one of the interacting molecules is coupled to a magnetic bead. Preferably, the target is linked to the first bead of the invention. It is understood however that arrangements wherein the capture molecule is coupled to one of the magnetic beads, or wherein different capture molecules are each coupled to a magnetic bead are also envisaged within the present invention.

According to aspects of the present invention the first bead can have a size which is smaller, equal, or even larger than the second bead. It is however preferred that, when the first bead is attached to a target biomolecule, the first bead is smaller than the second bead for several advantages which are explained further on in detail. The first and second bead can have the same composition. In this case, the magnetic moment which can be obtained is related to the size of the bead. However certain embodiments can be envisaged where the composition of first and second bead are different. In this case the correlation between size and magnetic moment is uncoupled.

The size of the first and second bead is not per se a limiting factor of the present invention. However, for detecting interactions on a biochip, small sized beads will be advantageous. When micrometer-sized beads are used as labels, they limit the downscaling because every label occupies an area of at least 1 μm². The resulting biochip size can be estimated as follows: On every capture spot, at least 1000 particles need to be collected to get a statistical counting variation of 3% (N^{-1/2}). To obtain a dynamic range of 1000, every spot needs an area of at least 1000×1000=10⁶ μm². A 1000-plex biochip will require a total area of 1000×10⁶ μm²=10 cm² for the capture spots, plus the open areas between the spots, etc. This is quite large for a biochip, which should be in the mm² range rather than in the cm² range to be cost-effective. Furthermore, due to steric hindrance micrometer-sized beads are not suitable as labels for assay conditions (e.g. target concentration, capture probe affinity, capture probe density on the surface) that would normally generate target concentrations on the surface higher than one per square micrometer. Furthermore, small particles have better diffusion properties and generally show a lower tendency to sedimentation than large particles.

According to the present invention the first bead is preferably in the size range between 1 and 500 nanometer, more preferably between 5 and 100 nanometer. The second bead according to the present invention is preferably in the size range between 50 nm and 5 micrometer. However, both beads can have the same size in the range 3 nm to 5 micrometer.

Where magnetic or paramagnetic beads are used in accordance with aspects of the present invention the application of a magnetic field can result in a repulsion and/or attraction between the first and the second bead (where both beads are magnetic) or movement of one of the beads due to a magnetic field gradient. The particles of the present invention which are used to make beads can be made of a magnetized or magnetizable material, but can also be solid or porous material such as polymers to which magnetic particles can be attached or incorporated. The use of magnet particles complexed with another material in beads makes it possible to uncouple the direct relation between magnetic moment and size of the bead. The use of magnet particle complexes in beads also allows modification of the weight of the bead. The use of magnet particle complexes in beads also allows the incorporation of functional groups for reaction with proteins and/or detection labels, e.g. chromophoric groups. Well known examples of beads comprising magnetic material are Dynabeads™. Dynabeads M450 (diameter 4.5 microns) can be coated with a monomeric epoxide, resulting in a mixture of epoxy and hydroxy groups. Dynabeads M-280 (diameter 2.8 microns) are polystyrene beads having hydroxyl groups which have been converted into tosyloxy groups by reaction with p-toluene sulphonyl chloride.

Magnetic beads are widely used in biological analysis, e.g. in high-throughput clinical immunoassay instruments, sample purification, cell extraction, etc. Several diagnostic companies (Roche, Bayer, Johnson & Johnson, Abbott, BioMerieux, etc.) fabricate and sell reagents with magnetic beads, e.g. for immunoassays, nucleic-acid extraction, and sample purification. Magnetic beads are commercially available in various sizes, ranging from nanometers to micrometers. For attachment or binding of the beads of the invention to the bioactive molecules, the beads may carry functional groups such as hydroxyl, carboxyl, aldehyde or amino groups. These may in general be provided, for example, by treating uncoated monodisperse, superparamagnetic beads, to provide a surface coating of a polymer carrying one of such functional groups, e.g. polyurethane together with a polyglycol to provide hydroxyl groups, or a cellulose derivative to provide hydroxyl groups, a polymer or copolymer of acrylic acid or methacrylic acid to provide carboxyl groups or an aminoalkylated polymer to provide amino groups. U.S. Pat. No. 4,654,267 describes the introduction of many such surface coatings. Other coated particles may be prepared by modification of the beads according to the U.S. Pat. Nos. 4,336,173, 4,459,378 and 4,654,267. For example, macroreticular porous polymer particles, prepared from styrene-divinylbenzene and with a diameter of 3.15 μm were treated with HNO₃ to introduce —NO₂ groups at the surface of the pores. Then the particles were dispersed in an aqueous solution of Fe²⁺. The Fe²⁺ is oxidized by the —NO² groups which leads to precipitation of insoluble iron oxy-hydroxy compounds inside the pores. After heating the iron exists as finely divided grains of magnetic iron oxides throughout the volume of the porous particles The NO₂ groups are reduced by the reaction with Fe²⁺ to NH₂ groups. To fill up the pores and to introduce the desired functional groups at the surfaces, different monomers are caused to polymerize in the pores and at the surface. In the case of a preferred type of particle, the surface carries —OH groups connected to the polymeric backbone through —(CH₂CH₂O) 8-10 linkages. Other preferred carry —COOH groups obtained through polymerization of methacrylic acid. For example, the NH₂ groups initially present in the beads may be reacted with a diepoxide as described in U.S. Pat. No. 4,654,267 followed by reaction with methacrylic acid to provide a terminal vinyl grouping. Solution copolymerization with methacrylic acid yields a polymeric coating carrying terminal carboxyl groups as in R452 beads referred to below. Similarly, amino groups can be introduced by reacting a diamine with the above product of the reaction with a diepoxide, while reaction with a hydroxylamine such as aminoglycerol introduces hydroxy groups.

The coupling of a bioactive molecule to a bead can be irreversible but can also be reversible by the use of a linker molecule for the crosslinking between particle and bioactive molecule. Examples of such linkers include peptides with a certain proteolytic recognition site, oligonucleotide sequences with a recognition site for a certain restriction enzyme, or chemical reversible crosslinking groups as those comprising a reducible disulfide group. A variety of reversible crosslinking groups can be obtained from Pierce Biotechnology Inc. (Rockford, Ill., USA).

According to a preferred embodiment of the present invention, detection of the specific interaction between a target and capture molecule (i.e. after removal of non-specifically bound molecules and/or labels) is done based on the properties of the particle which is linked to the biomolecule, which according to most embodiments of the invention is the first particle. The detection is preferably done based on the magnetic properties of the first particle.

According to aspects of the present invention, a magnetic field is applied on a sample comprising the first and second bead of the invention. Magnetic fields can be applied in several ways known to the person skilled in the art to provide different effects on magnetic beads as described in the present invention. A magnetic field should be large enough to generate an orientation of the magnetic moment in one or both particles, preferably saturating the magnetic moment in both particles. The required field depends on the type of particle (e.g. superparamagnetic, ferromagnetic, spherical or non-spherical). According to the present invention the field is in the range 1.10⁻⁴ Tesla to 10 Tesla, preferably in the range between 0,01 to 1 Tesla. Magnetic fields may be generated by permanent magnets or by electromagnets, e.g. solenoids. Variable fields can be generated by varying the current in magnet coils, but also by mechanical movement of magnetic material (hard-magnetic or soft-magnetic).

Magnetic field gradients can be generated near to current wires, but also by having curved or pointed shapes in magnetic material. Fields gradients applied to the magnetic particles range from 0.01 T/m to 10⁵ T/m, preferably between 0.1 T/m and 10⁴ T/m.

Detection of a bound target molecule or of a magnetic bead can be done by any suitable method, e.g. magnetically. For example, the detection may be performed by use of magneto-resistive sensors or by magnetic-induction methods etc, by mechanical methods (surface or bulk acoustic waves, crystal microbalance, vibrating membranes etc.), optical methods (surface plasmon resonance, optical interference, diffraction, surface-enhanced resonant Raman scattering, optical scattering, etc.) electrical methods (e.g. conduction, assisted by chemical development of the particles, etc.), or even other analytical tools such as mass spectrometry may be used. It is within the scope of the present invention that the beads are part of a composite detection label, e.g. containing additional optically active constituents, additional electro-active constituents, etc. Detection can occur optically (e.g. by fluorescence, evanescent-field excited fluorescence, fluorescence polarization, chemiluminescence, electrochemiluminescence, surface enhanced Raman scattering, etc.), electrically (e.g. via conduction, by redox currents, etc.), mechanically, etc. Special label designs are included within the scope of the present invention such as:

a magnetic core with a metallic cover layer, e.g. for enhanced stability, conductive properties, optical properties (e.g. scattering, plasmon resonance), etc.

magnetic core or magnetic cores surrounded by optically active constituents

a magnetic core or magnetic cores surrounded by (bio)chemically active molecules, e.g. enzymes, redox molecules, redox enzymes.

a magnetic core or magnetic cores coated with an organic layer, e.g. dextrane, to which bio-active and/or other functional compounds are coupled.

a magnetic core or magnetic cores encapsulated within a polymer sphere, e.g. polystyrene (commonly known as latexes), PMMA. Within the polymer matrix other signaling molecules (e.g. fluorophores) can be embedded or co-polymerized.

biologically active magnetic particles such as ferritin.

vesicles with magnetic and/or detection components, e.g. magnetosomes, liposomes.

Examples of technologies and applications of magnetic nanoparticles can also be found at recent conferences, such as “Bioconjugated nanoparticles in molecular diagnostics and therapy”, May 22-24, 2003, Jena (Germany). See www.ipht-jena.de/BEREICH_{—}3/molnano/nanoparticles2003/and the “2nd international meeting on the diagnostics applications of magnetic microspheres”, Paris, France, Jun. 12-13, 2003.

In another embodiment the first bead can carry an additional tag for detection, such as, but not limited to an antigen, a chromophore group, an affinity label. Tags for detection can easily be applied to a magnetic bead, when the beads harbor additional functional groups for reacting with detection tags as can be the case for polymer metal beads. Several techniques are known in the art to couple a tag to a bead, for example directly via a functional group, or indirectly via a linker or tether, or using intermediate molecules such as biotin/streptavidin.

### FIRST EMBODIMENT

According to a first embodiment of the present invention which is shown schematically in FIG. 1 detection of the interaction between biomolecules is performed, whereby one of the biomolecules C (preferably referred to as the capture molecule) is coupled to a surface 5 directly, e.g. when the surface 5 comprises a material such as a polymer to which the capture molecule binds or indirectly via a linker molecule 3. A surface 5 as used herein relates to a fixed substrate, matrix or grid suitable for coating directly (e.g. by cross-linking) or indirectly with biomolecules, such as glass, plastic, an organic crystal or an inorganic crystal (e.g. silicon), an amorphous organic or an amorphous inorganic material (e.g. siliconnitride, siliconoxide, siliconoxinitride, aluminumoxide). Suitable surface materials and linking chemistries are known to the person skilled in the art, and are described for instance in “Diagnostic Biosensor Polymers”, by A. M. Usmani and N. Akmal, American Chemical Society, 1994 Symposium Book Series 556, Washington D.C., USA, 1994, “Protein Architecture, Interfacing Molecular Assemblies and Immobilization Biotechnology”, edited by Y. Lvov and H. Möhwald (Marcel Dekker, New York, 2000), “The Immunoassay Handbook” by David Wild (Nature Publishing Group, London, 2001, ISBN 1-56159-2706) or “Handbook of Biosensors and Electronic Noses. Medicine, Food and the Environment” by Kress-Rogers (ISBN 0-8493-89054).

The invention can be performed on planar sensor surfaces (e.g. planar glass biochip), but also in a flow-through system (e.g. flow-through sensors made of porous aluminum oxide, porous silicon, or a porous column containing microbeads).

According to an aspect of the first embodiment, two beads 1, 2 each with a different magnetic moment, are used to modulate, e.g. minimize non-specific binding in the detection of a capture molecule-target molecule interaction as shown schematically in FIG. 1. One or a mixture of biomolecules comprising known or putative target(s) T is linked (directly or indirectly) to the first bead 1. The first bead 1 has a smaller magnetic moment than the second bead 2. The surface 5 is coated with one or preferably a selection of capture molecules C. The coated surface is contacted with the target/mixture together with or before adding the second bead 2 with the larger magnetic moment, itself not being coupled to a biomolecule. Application of an adequate magnetic field M (the Fig. shows an out-of-plane magnetic field, but this is not essential) will result in attraction of the first bead 1 to the second bead 2. The magnetic moments of the first and second bead 1, 2 and the strength of the magnetic field M are selected so that a weaker or non-specific interaction of a labeled biomolecule T with the capture-molecule C or the surface 5 results in removal of the molecule T, i.e. disruption of the binding between the target and capture molecules T, C. The freed target molecule can be moved away. On the other hand, the magnetic moments of the first and second bead 1, 2 and the strength of the magnetic field M are selected so that a stronger, e.g. specific target-capture molecule interaction will not be disrupted by the magnetic attraction between the two beads 1, 2, and can be detected.

Several applications of this embodiment are included within the scope of the present invention. For example, a collection of structurally related analogues of a known ligand or a large collection of small compounds (capture molecules) can be applied to a surface, whereby a single protein (target molecule) can be used for assaying weak, moderate and strong binding of the protein to a ligand analogue. This allows the discovery of putative lead compounds for agonists and antagonists for the assayed protein. A similar assay can be used with several proteins. Herein, proteins which bind weakly or strongly will give indications for possible side effects.

Similar assays can be performed wherein the protein component is attached to a surface and the small compounds are in solution, depending on whether it is easier to link to small compounds to the surface or to the magnetic bead.

This embodiment of the present invention can also be used for protein engineering. A collection of site directed or random mutagenized proteins can be spotted on a grid surface and screened for binding of a certain compound (small molecules or other protein or proteins). With this arrangement, proteins can be determined having small or modified binding affinity with its normal ligand, but will also reveal mutants which show modified binding characteristics or a modified ligand specificity. Following the teaching of the preceding examples, analogous experiments can be designed for interactions between proteins and DNA, RNA, carbohydrates, lipids, phosholipids, other cellular components. Also cells, vesicles, pathogens, and other biological bodies can be detected. This embodiment of the present invention is suitable for the detection of a bioactive molecule but also for the isolation and purification of a bioactive molecule.

A preferred type of interactions to be determined in accordance with this embodiment of the present invention are those where both the capture and the target molecule are proteins. An application hereof is a so called protein array wherein a collection of known proteins (capture molecules) are coupled to a grid. A biological sample comprising target molecules is labeled with the first magnetic beads and assayed for the binding between target molecules and capture molecules. The second particles are then used to identify or remove those target molecules which are more weakly e.g. a-specifically, bound to the capture molecules.

The present invention allows to perform this method without the need of changing the buffer conditions in order to discriminate between specific and a-specific binding.

When working in a protein microarray, the size of the first magnetic bead can be of importance. Micrometer-sized magnetic beads have been used as labels in an array sensor (e.g. Baselt D. R., Lee G. U. et al., ‘A biosensor based on magnetoresistance technology’, Biosensors and Bioelectronics 13 (1998) 731-739). Although magnetic forces can easily be applied to large beads due to the large magnetic moment, large beads have many important disadvantages in an experiment such as a protein-protein microarray:

To have a good chance of binding to the surface, the labels need to interact with the sensor surface during a considerable time. The interaction efficiency of the beads with the surface is for a good part determined by the diffusion speed of the labels. The diffusion of micrometer-sized particles is very slow (D˜10⁻¹² m²/s, about 1 μm in 1 second), which increases the total assay time.

The slow diffusion speed, long interaction time with the surface, and the large contact area of the particles increases the chance that the large beads stick to the surface in a non-specific manner.

Micrometer-sized particles show a sedimentation speed which is detrimental to the assay.

Micrometer-sized particles are sensitive to fluid flow. During an assay, solutions are often refreshed or stirred (e.g. during incubation) and different fluids can be applied in succession. When large beads are used as labels, these can be ruptured from the sensor in an uncontrolled way by such fluid movements. As a consequence, micrometer-sized labels can only be applied for endpoint detection, and even in that case fluid manipulations need to be performed with great care.

Small beads can be dispersed into a fluid at higher concentrations than large beads (in terms of number of beads per unit volume). Thus, small beads result in a higher interaction rate with the target molecules and sensor surface.

Thus, it is advantageous to use beads that have a size below a micrometer. Thus, according to a preferred embodiment of the invention, sub-micrometer magnetic first beads are used, in size the range of 500 nanometers down to a few nanometers.

When using small particles, the problem arises that the magnetic moment of a bead decreases. The moment scales with the volume of the bead, so with the third power of the radius of the bead. For example, superparamagnetic beads with a size of 35 nm and a magnetic core of about 10 nm, have a magnetic moment of the order of 10⁻¹⁸ A.m². Even with a large external gradient of 10³ T/m, a force of only 1 fN [Eq. (1)] is obtained. This is largely insufficient to separate weak from strong binding between bioactive molecules. Therefore, these small particles can only be used when the field gradient is strongly increased.

A known way to increase the magnetic gradient is by using magnetic materials with very strong curvatures or pointed shapes. For example, a magnetic needle with an apex curvature of 100 micrometer generates a field gradient of about 1 T/100 micrometer=10⁴ T/m at the apex (given that the saturation magnetization of magnetic materials is about 1 T). However, this gradient rapidly falls off, particularly when moving further than 100 micrometer away from the apex. In other words, high field gradients are always spatially inhomogenous and are applied only locally. Therefore, the magnetic material generating the field gradient should be brought as close as possible to the first beads that need to be actuated. Meanwhile, the gradient generation method should not perturb the fluid flow across the sensor (fluid channels typically have a height of 50 micrometer and higher) and allow for simple control of the size and orientation of the field and the field gradient in time.

This invention proposes in one aspect a new method to apply a magnetic force to first beads, namely by dynamically approaching the first beads by second magnetic beads inside the fluid. The magnetic moment of the second beads generates a large magnetic field gradient locally on the first beads. This translates into a strong magnetic interaction between the first and second beads. Meanwhile, due to their larger magnetic moment, the second beads can be magnetically actuated with relatively small field gradients, or they can be actuated by different methods.

The magnetic moments of the particles will determine the forces between the beads and hence the stringency. In order to gradually remove labeled capture molecules with a increasing binding strength between capture and target molecule, the methods of the present invention can be performed with consecutive washing steps with at least one type of third magnetic particles with increasing magnetic moment, these moments being larger than the moment of a second magnetic bead. This gradual washing also enables to estimate within narrower ranges the binding strength between a capture and a target molecule. Another way to generate gradual removal of increasing binding strengths, is by consecutively applying larger external fields while using a bead of which the magnetic moment increases with the applied field.

According to the above embodiment it is possible to re-use the second beads. They can be made to approach the first beads, then remove weakly bound first beads, then move away from the initial point of approach, and subsequently can be re-used to remove weakly bound first beads elsewhere on the surface.

According to the above embodiment it is also possible to re-use the first beads linked to those bioactive molecules which are not bound specifically and are removed by binding to the second bead.

Target detection according to the above embodiment can be designed in several ways:

The targets can be labeled by coupling them with small magnetic beads before the targets bind to the sensor surface. The pre-labeling can be performed in a generic way (e.g. the beads have a generic protein-binding chemistry on their surface) or with bioactive molecules with a specific binding for capture molecules (e.g. antibodies that carry a magnetic bead).

Small magnetic beads can be attached to the targets after these have bound to the sensor surface, again in a generic or specific way.

The assay can be a binding assay, competition assay, displacement assay, etc.

The application of magnetic forces to a multi-molecular complex increases the chance of molecular dissociation. This can help to increase the speed of a displacement assay, which is normally very slow due to the high affinity and low dissociation rate of capture molecules.

### Example 1 of the First Embodiment

determination of forces between magnetic beads.

Several combinations of beads are possible. A good balance needs to be sought between the size (the smaller the better) and the magnetic properties (magnetic moment, magnetic relaxation) of the beads. This example presents a comparative study on difference parameters for two pairs of particles according to a preferred embodiment of the invention.

In the following we will consider two situations:

In the following examples, the first and second bead differ in size and magnetic moment accordingly. For practical reasons, the first bead and second bead of the invention will be referred to as the ‘small’ and ‘large’ bead.

Example A

First (small) beads: diameter 100 nm, m=10⁻¹⁶ A.m², superparamagnetic

Second (large) beads: diameter 1-μm m=10⁻¹³ A.m², superparamagnetic

Example B

First (small) beads: diameter 35 nm, m=10⁻¹⁸ A.m², superparamagnetic

Second (large) beads: diameter 100 nm, m=10⁻¹⁵ A.m², high-density magnetic material

In the numerical examples, m denotes the saturated magnetic moment of the beads. The examples have been selected such that the second beads are able to generate a large field gradient on the first beads, generating a force between the two beads higher than 0.1 pN. For the following calculations the particles are approximated to be spherical.

Force between large and small beads

To calculate the attractive force between a first and a second bead, the force between two magnetized beads is determined by the size of the magnetic moments, the relative orientations of the moments, and the relative position of the beads. For a bead-to-bead approach along the direction of the externally applied magnetic field (pole-to-pole approach), the attractive dipole-dipole force is given by:

$$F = \frac{{μ}_{0}}{4 π} \frac{6 {m}_{1} {m}_{2}}{{x}^{4}} \qquad Eq . ( 2 )$$

where m₁ and m₂ are the moments of the respective beads and x is the center-to-center separation.

The maximum magnetic force that a second bead can exert on a first bead occurs when the beads are in closest contact.

For example A an attractive force is calculated of 46 pN for a surface-to-surface separation of 50 nm (x=0.6 μm) and 66 pN in case of contact (x=0.55 μm).

For example B an attractive force is calculated of 17 pN for a surface-to-surface distance of 10 nm (x=77.5 nm) and 29 pN in case of contact (x=67.5 nm).

Thus, the method according to this embodiment provides the possibility to distinguish between stronger and weaker biomolecular interactions with a threshold force of the order of tens of piconewtons.

Velocity of the Large Beads

Due to their large magnetic moment, the second beads can be manipulated inside the fluid by externally applied magnetic field gradients. The manipulation speed for a second particle (large bead) can be estimated by using the equation for the flow resistance of a spherical particle in a viscous medium:
F=6πηrv Eq. (3)
with η the viscosity of the fluid, r the radius of the particle, and v the particle speed with respect to the surrounding fluid far away from the bead.

With a gradient of 10³ T/m, the second beads of the example A experience a force of 100 pN [Eq. (2)] and a velocity of 10 mm/s [Eq. (3)]. At the same gradient, the second beads of example B have a force of 1 pN and a velocity of 1 mm/s. This means that the second beads within a range of 100 nm to 1 μm can be moved rapidly (mm/s) towards and away from the sensor surface when a magnetic field is applied. Also, this range in size and magnetic moment can be exploited to obtain forces which are sufficient to obtain a discrimination between weaker and stronger, e.g. specific and a-specific, binding in the methods of the present invention (see also embodiments 2 and 3).

Repeated Stringency

It can be shown that first and second beads in solution hardly affect each other when they are both present in solution:

(i) In the two given situations, the ratio of the magnetic moments of the first and second beads is 10 or more e.g. 10³. As a consequence, the magnetic forces on the respective isolated beads and their velocities also differ by several orders of magnitude. This means that the second beads can be magnetically moved towards and away from the sensor while the first beads in solution are hardly actuated.

(ii) The first beads feel a significant particle-to-particle force only when a second bead comes into very close proximity [the force decreases like x⁻⁴ in Eq. (2)]. Such a close approach is easily achieved at a surface where the first beads are immobilized, but has a very low probability when both beads are in solution.

As a result, the two-bead stringency procedure can be repeated, regularly removing loosely bound first beads from the surface. Then, the sensor status can be regularly monitored rather than only polled at the endpoint of the assay. The resulting recording of dynamics and kinetics on the sensor gives the advantage of improved reliability, precision and speed of the biological measurement.

Lateral Manipulation of Large Beads

Until now it has been assumed that the second beads are moved towards and away from the sensor surface with their trajectories essentially perpendicular to the surface. The efficiency with which second beads pick up weakly bound first beads can be increased by also creating a lateral movement of the second beads over the sensor surface. The lateral movement can be created by any suitable means of which shear flow of the fluid, acoustic excitation, or magnetic actuation are only examples. The latter can be generated with external field gradients as well as through gradients created on the chip, e.g. by passing a current through on-chip current wires. On-chip current wires have the advantage that the gradient is generated very close to the binding surface with very little energy consumption. The magnetic field gradient generated around an on-chip current wire equals:

$$\frac{ⅆ B}{ⅆ R} = \frac{{μ}_{0} I}{2 π {r}^{2}} \qquad Eq . ( 4 )$$

with I the current through the wire and r the distance from the wire. As an example, a current of 10 mA at a distance of 10 μm generates a field gradient of 20 T/m. Assume that the beads are magnetically saturated by an externally applied uniform magnetic field and that an additional non-uniform field is created by the on-chip current wires. The gradient of 20 T/m gives a velocity of 0.2 mm/s for the large beads of example A, and a velocity of 21 μm/s for the second beads of situation B. When the sensor has a width of 10 μm, the second beads can be made to move across the sensor many times or several times per second. This lateral movement can increase the exposure of sensor surface to the second beads and increase the chance of picking up weakly bound first beads.

Aggregation of Large Beads

This two-bead stringency embodiment is relatively insensitive to magnetic aggregation, for two reasons:

(i) The first, e.g. small beads with a lower magnetic moment can have little tendency to aggregate in a magnetic field.

(ii) The stringency force on an immobilized first bead is determined by the single nearest second bead [cf. x⁻⁴ in Eq. (2)]. Forces from beads further away are negligible, so a potential clustering of second beads does not change the forces exerted on the first beads.

However, very large aggregates of the second beads (with a higher magnetic moment) are preferably avoided because the aggregates can have a reduced accessibility to the sensor surface. Aggregation can be reduced by switching the magnetic field off part of the time, by using beads with a very low remnant moment (e.g. due to fast magnetic relaxation, small magnetic domains, super-paramagnetism), by using anti-stick coatings, by fluidic shear stress, by using moderate concentrations of second beads, and by changing the orientation of the magnetic field (e.g. using varying magnitude and direction of magnetic field).

Note that the detection is performed on the target molecules or on the first beads. To avoid false signals from second beads that could be present in the sensitive detection zone, the second beads preferably do not generate signal contributions during the measurement. For example, the detection is done via first beads with fluorescent tags, while those tags are not present on the second beads.

### Example 2

A stringency procedure according to a preferred embodiment of the invention will be described with reference to FIG. 5. The device of FIG. 5 may be implemented as a microfluidic device.

1. First beads 1 with a lower magnetic moment and optionally smaller size are attached to target molecules and provided in source 11. Under the control of valve 1 and pump 13 these are introduced into a measuring chamber 15 in which there is a substrate 16 with capture spots, i.e. capture molecules coupled to the sensor surface of substrate 16. The transport of beads toward the surface can be enhanced, e.g. by fluid flow, stirring, or by application of a magnetic field gradient.

2. The first beads 1 attached to the target molecules are immobilized on the sensor surface by their binding to capture molecules. A modification of this embodiment is the provision of different capture spots on a biochip 16 having different strengths of the interactions between bioactive molecules. Therefore, it may can be advantageous to select first beads 1 with a somewhat lower magnetic moment for capture spots that have weak bio-molecular interactions, and first beads 1 with a somewhat higher magnetic moment for capture spots that have strong interactions.

3. Second beads 2 with a larger magnetic moment are supplied from source 10 by operation of valve 2 and pump 13. The second beads may be optionally of larger size than the first beads 1. Second beads 2 are moved towards the sensor surface where the first beads 1 are immobilized. The movement can be driven by magnetic forces, e.g. generated by a magnetic field generator 14 such as one or more permanent magnets or an electromagnet. Flow of a liquid can also be provided from a source 12 by operation of valve 3 and pump 13. For optimal control of the trajectories of the second beads 2, the magnetic forces as well as a forced flow of the fluid above the sensor surface should preferably be taken into account and synchronized.

4. When the second beads 2 approach the first beads 1, the first beads 1 experience an attractive magnetic force towards the second beads 2. Weakly bound first beads 1 detach from the sensor surface, are attracted toward the second beads 2 and become magnetically attached thereto. First beads 1 that are strongly bound to the sensor surface stay on the sensor surface.

5. It is possible that first-bead-to-second-bead complexes form due to magnetic attraction, but that the two-bead complexes do not immediately move away from the sensor surface. In that case an additional excitation can be applied to remove the complexes from the surface, e.g. a magnetic field gradient that pulls the formed complexes away from the surface, a shear flow that generates shear forces along the sensor surface, ultrasonic excitation, etc. These excitations remove complexes of which the first beads are weakly bound to the surface.

6. The second beads 2—with or without first beads 1 attached—are moved away from the sensor surface, e.g. by washing using the fluid in source 12, to exit 19. As a result, the first beads 1 that were initially weakly bound to the sensor surface, are now removed from the sensor.

7. The first magnetic beads 1 which now remain attached to the capture molecules may be sensed by any suitable technique, e.g. by magnetic sensors 17 located underneath the substrate 16 using sensor circuitry 18. If the first beads 1 are marked with optically active material, e.g. a dye or a fluorescent material, the presence of the first beads 1 may be detected optically. If the first beads 1 are marked with a material which alters electrochemical potential or enables electrochemical charge transfer, the presence of the first beads may be detected by using suitable electrodes. If the beads contain radioactive material they can be detected by radioactive emissions.

The above sequence can be repeated several times with the same set of second beads 2. The method can be applied either in well plates (e.g. microtiter plates) or in microfluidic cartridges. Well-plates are highly suited for robotized high-throughput applications; cartridges allow a high degree of functional integration and fluidic miniaturization around the sensor.

### SECOND EMBODIMENT

According to a second embodiment of the invention shown schematically in FIG. 2, two beads 1, 2 are used, which are each coupled to a bioactive molecule. The beads 1, 2 optionally have the same magnetic moment. In one experimental arrangement, the two beads 1, 2 can be coupled respectively to a target T and a capture C molecule. In an alternative arrangement the two beads 1, 2 are coupled to two different target molecules T1, T2 which bind to different parts of a capture molecule C (see FIG. 3). For example, the two different target molecules T1, T2 can be antibodies, each either monoclonal or polyclonal, directed against different epitopes of the same antigen that is the capture molecule.

In both arrangements, the capture molecule C, which is either coated on a surface or in solution, is brought into contact with one or both bead-coupled target molecules T1, T2, which will bind to their respective binding sites or epitopes. However, other combinations of beads 1, 2 can also be created due to non-specific binding. Application of a magnetic field M with rapidly varying direction of its magnetic vector will cause magnetic repulsions or attractions between the two beads 1, 2, e.g. depending on whether the instantaneous magnetic field is perpendicular to the axis joining the two beads or parallel thereto. The sizes of the magnetic moments of the two beads 1, 2 and the magnitude of the varying magnetic field M are chosen so that in the case that one or both of the target molecules T1, T2 are not bound specifically, the attraction and repulsive forces result in removal of that molecule T1, T2, i.e. the bead-bead interaction is disrupted. The sizes of the magnetic moments of the two beads 1, 2 and the magnitude of the varying magnetic field M are also chosen so that in the case that the one or more target molecules T1, T2 are bound specifically, e.g. two polyclonal or monoclonal antibodies specifically bound to their respective epitopes, they will not be removed. The presence of both beads 1, 2 together in close proximity can be detected, e.g. optically. Due to the fact that a combination of two beads in close proximity can only occur if two selective bindings are present, e.g. two antibody bindings plus the control of stringency by application of the varying magnetic field, the presence of closely bound beads 1, 2 is a clear indication of the presence of the capture molecule C. In particular, when polyclonal antibodies are used, the chance of a-specific binding or cross-reactivity to other epitopes is increased and the application of the varying magnetic field, and thus a force on the binding, can be used to eliminate a binding which is not as specific as the desired binding. Thus, in the case of using two coupled antibodies as target molecules T1, T2, specific detection of an antigen (capture molecule C) can be obtained without the need for purified monoclonal antibodies, i.e. without the need for high levels of specificity.

The magnetic field can result in attractive forces, i.e. pushing two beads 1, 2 to each other, or can be repulsive, pulling the two beads 1, 2 away from each other. When both beads 1, 2 are coupled together via the target and capture molecules T1, T2, C, they are very close together and when a magnetic field perpendicular to the axis of the two beads is generated, the beads 1, 2 repel each other. Since the magnetic beads 1, 2 tend to align in a magnetic field and have generally random orientations in a fluid at the start, the magnetic field has to be constantly changed to obtain repulsion. Varying the direction of the magnetic vector can be done by a rotating magnetic field, e.g. by the provision of three coils orthogonal to each other with a current controller for controlling the phase and magnitude of AC currents in the coils to generate a magnetic field rotating rapidly or moving randomly or pseudo-randomly in three dimensions. This magnetic field should be large enough to generate an orientation of the magnetic moment in both beads 1, 2, preferably saturating the magnetic moment in the beads. The required field M depends on the type of particle (e.g. superparamagnetic, ferromagnetic, spherical or non spherical bead). The potential energy, U, of dipolar interactions between two magnetic beads 1, 2 is given by:

$$U = \frac{{μ}_{0}}{4 π} \frac{{{m}^{->}}_{1} \cdot {{m}^{->}}_{2} - 3 ( {{m}^{->}}_{1} \cdot {r}^{->} ) ( {{m}^{->}}_{2} \cdot {r}^{->} )}{{r}^{3}} \qquad Eq . ( 5 )$$

where \vec{m}₁, \vec{m}₂ represent magnetic moments of each bead, respectively r=distance between the bead cores.

The bead-to-bead force F is given by the gradient of the energy:
F=−∇U Eq. (6)

The magnetic force depends on the relative orientations of the bead moments (which are influenced by the externally applied field) and the bead-to-bead axis. When the bead moments are mutually aligned and oriented perpendicular to the bead-to-bead axis, there is a mutually repulsive force of magnitude:

$$F = \frac{{μ}_{0}}{4 π} \frac{3 \vert {m}_{1} \vert \vert {m}_{2} \vert}{{r}^{4}} \qquad Eq . ( 7 )$$

When the bead moments are mutually aligned and both oriented parallel to the bead-to-bead axis, then there is a mutually attractive force of magnitude:

$$F = \frac{{μ}_{0}}{4 π} \frac{6 \vert {m}_{1} \vert \vert {m}_{2} \vert}{{r}^{4}} \qquad Eq . ( 8 )$$

The above embodiment may be used for assays which require removal of capture molecules from the body. An example, is the removal of proteins from the mouth. Target molecules T1 labeled with first magnetic beads are introduced into the mouth and are allowed to complex with proteins in the oral cavity. These complexes are then removed from the mouth using a magnet to scavenge for all of the beads. Second magnetic beads labeled with a further target molecule T2 are then added to the recovered first beads and complexes between the two beads are allowed to develop. Then a rapidly rotating magnetic field is applied in order to disrupt first bead-second bead complexes that are not bound tightly together. The remaining complexes of both beads have specific bindings. The presence of remaining first and second bead complexes may then be determined by any suitable means, e.g. optically.

### THIRD EMBODIMENT

According to a third embodiment of the invention, two beads 1, 2 are used, wherein only one of them has a magnetic moment—see FIG. 4. An a-specific binding between two bioactive molecules C, T is disrupted by the combination of (1) a fluidic force generated on the non-magnetic bead 2 being attached to one molecule (either C or T but shown as the capture molecule C in FIG. 4) by placing it in a flow F of a liquid and (2) an opposing magnetic force which acts on the magnetic bead being attached to the other molecule by application of a magnetic field gradient MG. This application also allows to perform the present invention in solution, without the need of attaching a capture molecule C to a solid substrate such as a microarray.

Non-magnetic beads are available commercially. Any suitable non-magnetic material can be used, e.g. solid or semi-solid materials. Examples, include latex, polystyrene, cross-linked dextrans, methylstyrene, polycarbonate, polypropylene, cellulose, polyacrylamide, dimethylacrylamide.

For example, with a non-magnetic bead 2 with a radius of 0,5 μm and the fluid being water with a viscosity of η=1.10⁻³ Pa·s and the water flow v=about 1 mm/s, a fluid friction force of 9,4 pN is applied on the non-magnetic bead 2. With a magnetic bead 1 where m=1.10⁻¹⁵ Am² and the magnetic field gradient MG is dB/dz=1.10⁴ T/m a magnetic force of 10 pN is applied on the magnetic bead 1 (assuming that the magnetic bead is insignificantly small and is not affected by the fluid flow). Thus, by manipulating the magnetic moment of the bead 1, sizes of the magnetic and non-magnetic beads 1, 2 and flow of the liquid, a situation can be created wherein counteracting fluid force and magnetic forces are in balance. When the binding between the bioactive molecules T, C is strong (e.g. specific), the complex will remain at the same position in the liquid flow. In the case of weak (e.g. a-specific) binding, the binding will be disrupted by the tension induced by the opposing forces and the bioactive molecules C, T will either migrate towards along the magnetic field, or will float with the liquid flow.

This assay can also be performed with biological cells, viruses or other biological bodies, e.g. liposomes or vesicles. These bodies can be part of a biological complex that is put under compressive or tensile stress, or the bodies themselves can function as the second bead. Hence, in accordance with aspects of the present invention it is not necessary to use beads but use may be made of any suitable particles including biological bodies.

The exact position of a complex of bound capture and target molecule C, T in the liquid flow can be determined or measured, for example, optically. One example is the use of chromophoric groups on the magnetic and/or the non magnetic bead which allow optical detection of the presence of the beads in a particular location of the apparatus. Flow and/or magnetic field can be adapted in order to hold the complex at a fixed position in the fluid flow or to move the complex at a certain speed in a certain direction. With this embodiment it is possible to purify and/or enrich capture-target molecule complexes with specific binding.

Alternatively, the system can be used to fractionate a sample comprising capture and target molecules C, T coupled to magnetic and non magnetic beads 1, 2. Due to the size chosen, a large non-magnetic bead will encounter a larger fluid force than a smaller magnetic bead 1. After contacting a sample with coupled target and capture molecules C, T, a magnetic field gradient is applied which attracts bound and unbound target molecules, while other molecules remain in place and can be removed. When an increasing opposite fluid force is applied afterwards, firstly a-specific capture-target binding will be disrupted, and capture molecules will removed with the flow, secondly specific bound complexes will be removed with the flow while unbound target molecules will remain attracted by the magnetic field.

In a modified version of this embodiment, different types of capture molecules are used, each with a different size of non-magnetic beads. Flow rates and/or magnetic fields are manipulated in order to segregate the target-capture molecules complexes in accordance to the size of the non-magnetic bead.

The above described identification and/or purification of bound molecules can be performed in any system where a fluid flow and a magnetic field can be generated; magnetic fields can be generated externally or internally (e.g. coils or magnetic material). The volume of a sample can be reduced to the microliter range with detection region volumes down to 1 nl using for example a mesoscale flow system as described in U.S. Pat. No. 5,866,345, for instance.

### Example of the Third Embodiment

An example of the third embodiment will be described with reference to FIG. 6 which may be implemented as a microfluidic device. A hydromagnetic bottle is provided in zone 28. Fluid from source 20 is caused to flow, e.g. by means of controllable pump 21, through zones 27, 28, 29 and to exit from 30. Zones 27 and 29 are shaped so as to increase and decrease the flow, respectively, compared to the flow rate in zone 28. A magnetic field gradient is provided in zones 27-29 by a suitable magnetic field generator 24, e.g. one or more permanent magnets or electromagnets. The magnetic field attracts magnetic beads in a direction towards the left in FIG. 6. The flow of liquid from source 20 tends to drive a bead towards the right in FIG. 6. First magnetic beads 1 (typically very much smaller in size than the second non-magnetic beads 2) are labeled with target molecules and are brought into contact with the second non-magnetic beads 2 labeled with a capture molecule. The result is a mixture of beads 1, 2 some of which are bound together by biomolecular binding. When the forces are balanced, the combinations of first and second beads attached to each other via at least one biomolecule remain stationary, e.g. in zone 28 where they can be detected by a suitable detection system, e.g. an optical or magnetic system. The mix of beads and biomolecules is injected at 25 e.g. into zone 27. Initially, the flow of liquid from source 20 may be low or the liquid may be stationary. The effect of the magnetic field gradient will be to drag the magnetic first beads 1 towards the left in FIG. 6. To prevent the beads 1, 2 going too far, an optional filter 23 may be provided. The pump 21 is then activated to slowly raise the flow rate. Initially, non-magnetic beads 2 and other debris not coupled to the first beads 1 will be flushed out of the system via exit 30. When the fluid flow force of bead 1, 2 combinations reaches a high enough level these combinations will move to the right. If they enter zone 29, the flow rate reduces and the force generated by the magnetic field gradient will dominate preventing the beads from reaching exit 30. If the flow rate is slightly too low, the magnetic field gradient will tend to move some of the bead combinations 1, 2 towards the left in FIG. 6. However, as there is a higher flow rate in zone 27, the fluid flow force will dominate and prevent the combinations from going further. The result is that bound combinations of beads 1, 2 will be trapped in the hydromagnetic bottle 28. Here, the binding between bead 1 and/or bead 2 will be placed under stress caused by the opposing magnetic and fluid forces. If the binding is non-specific, the binding will be disrupted and the magnetic first beads 1 will move towards the filter 23 and non-magnetic particles will be flushed from the system. Hence, bead combinations which remain in the hydromagnetic bottle 28 will have specific bindings.

1. Use of a first and a second particle, at least one of which is magnetic, in a magnetic field for distinguishing between different strengths of bindings between microbiological entities in a liquid, the use comprising:
providing a complex between a first particle mobile in the liquid and a first microbiological entity,
providing conditions within the liquid for a binding between the first microbiological entity and a second microbiological entity;
bringing a second particle mobile in the liquid into proximity with the complex; and
acting on the first and/or second particle to apply a mechanical stress to the binding between the first and second microbiological entity while applying the magnetic field to thereby disrupt a binding of a first strength and not to disrupt a binding of a second greater strength.

2. Use according to claim 1 wherein the distinguishing of the strength of a binding is used for the discrimination between a specific and an a-specific binding.

3. Use according to claim 1, wherein the first microbiological entity is a target molecule and the second microbiological entity is a capture molecule.

4. The use according to claim 1, wherein both first and second particles are magnetic particles.

5. The use according to claim 1 wherein a first particle is coupled to a microbiological entity and wherein a second magnetic particle is not coupled to a microbiological entity.

6. The use according to claim 1 wherein both the first and the second particles are coupled to a microbiological entity.

7. The use according to claim 6 wherein the first particle is coupled to a target microbiological entity and the second particle is coupled to a capture microbiological entity.

8. The use according to claim 6 wherein the first particle is coupled to a first target microbiological entity and wherein the second particle is coupled to a second target microbiological entity.

9. The use according to claim 4 wherein the first and/or second magnetic particles is paramagnetic.

10. The use according to claim 4 wherein the first magnetic particle has a magnetic moment which is 10 times smaller than the magnetic moment of the second magnetic particle.

11. The use according to claim 4 wherein the size of the first magnetic particle is smaller than the size of the second magnetic particle.

12. The use according to claim 4 wherein the first magnetic particle has a diameter between 1 nm and 1 μm, more preferably between 10 nm and 200 nm.

13. The use according to claim 4 wherein the second magnetic particle has a diameter of at least 100 nm.

14. The use according to claim 1 wherein the first or second microbiological entities are arranged on capture spots on an array.

15. The use according to claim 1, wherein only one of the first and second particles is magnetic and the other particle is non-magnetic.

16. The use according to claim 15 wherein the non-magnetic particle is larger than the magnetic particle.

17. The use according to claim 1 further comprising the step of applying a fluid frictional force to the first or second microbiological entity.

18. A tool for the distinguishing between bindings of different strengths between microbiological entities, the tool comprising:
first particles and second particles, at least one of which is magnetic,
means acting on the first and second particles to thereby exert a mechanical stress on bindings between the first and second microbiological entities and to distinguish between the bindings of different strengths, the means for exerting a mechanical stress comprising at least a magnetic field generator.

19. A tool according to claim 18 wherein both first and second particles are magnetic or the first particles are magnetic and the second particles are not magnetic.

20. The tool according to claim 18, wherein first and/or second particles are coupled to a microbiological entity.

21. The tool according to claim 18 wherein the microbiological entity is a bioactive molecule such as a protein or a peptide.

22. The tool according to claim 18, wherein the means for exerting a mechanical stress includes means for exerting a fluid frictional force on the first or second particles.

23. The tool according to claim 18, further comprising an array of microbiological entities arranged on capture spots on a substrate.

24. The tool according to claim 18, further comprising means for generating an excitation that forces a lateral movement of the particles with respect to the array.

25. Use of the tool according to claim 18 for the identification, isolation, purification of a specific bound bioactive molecule.
